(19) United States
(12) Patent Application Publication
(10) Pub. No.: US 2001/0036460 A1
(43) Pub. Date: Nov. 1, 2001

(54) Targeted death cancer cells

(76) Inventor: Janet A. Sawicki, Newtown Square, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/828,409

(22) Filed: Apr. 6, 2001

Related U.S. Application Data
(60) Provisional application No. 60/195,179, filed on Apr. 6, 2000.

(51) Int. Cl. A61K 39/395

(52) U.S. Cl. 424/178.1

(57) ABSTRACT

The present invention relates to the use of an inducible genetic recombination system to target expression of a toxin to cancer cells and to hereby prevent the recurrence of particular tumors. The present invention also relates to the use of an inducible genetic recombination system to target expression of diphtheria toxin to androgen-independent cancer cells following androgen ablation therapy and to thereby prevent the development of recurrent prostatic tumors.

### CROSS REFERENCE TO RELATED APPLICATIONS

This application claims priority under 35 U.S.C. § 119, based upon U.S. Provisional Patent Application No. 60/195,179, filed Apr. 6, 2000.

### FIELD OF THE INVENTION

The present invention generally relates to the fields of medicine and oncology and to a method of utilizing an inducible genetic recombination system to prevent the development of cancer and, more particularly, to the use of such an inducible genetic recombination system to target expression of diphtheria toxin or other toxins to androgen-independent cancer cells following androgen ablation therapy or to target expression of diphtheria toxin or other toxins to other types of cancer cells, for which the regulatory sequences of a molecular marker expressed in the cancer cell have been identified.

### BACKGROUND OF THE INVENTION

The standard therapy for men with metastatic prostate cancer is androgen ablation by either bilateral orchidectomy or the use of luteinizing hormone-releasing hormone analogues. Initially, a favorable response is seen in 70-80% of patients when serum testosterone is reduced to castrate levels. Inevitably, however, patients who exhibit an initial therapeutic response succumb to androgen independent carcinoma within three years. Hormone refractory disease is most likely the result of clonal expansion of some prostate cancer cells that are able to escape the growth-restraining effects of low circulating androgen levels. The underlying molecular mechanism(s) by which some prostatic cancer cells become androgen independent remains largely unknown. Recent studies suggest that acquired apoptosis resistance resulting from the upregulation of the protooncogene bcl-2 (10,16,26,28,33) and/or caveolin (35) may play a role in tumor cells becoming androgen independent. Other studies point to changes in the androgen signaling axis as the mechanism (11-13,24,25).

Targeted death of androgen-independent prostate cancer cells is an attractive therapeutic option. The idea of directing plant or bacterial toxins to specific cells for the treatment of cancer has been suggested and even shown to be effective in cultured cells (27,31,32,34). There has been slow progress, however, in introducing this strategy into the clinical setting. This has been due in large part to the inability to tightly control the activation of cell specific regulatory elements that direct the expression of highly toxic genes. Even very low levels of basal transcriptional activity would result in unwanted and harmful cell death in the patient.

Very recently, new genetic strategies that are well suited for the delivery of toxin genes have been developed. Using these strategies, the expression of genes is tightly regulated in an inducible and cell-specific manner. Gene expression in these systems is not based on transcriptional regulation, but rather on regulated recombination (5,6,14,41). While each of these systems is slightly different from the other, they all rely on the fusion of a ligand binding domain (LBD) from a steroid hormone receptor to a site-specific recombinase, for example Cre or Flp recombinase (FIG. 1).

Activity of the recombinase is dependent upon ligand administration. Upon activation of the recombinase, a gene sequence containing the target sequences for the recombinase undergoes recombination, thereby becoming functional. The recombination event only takes place in selected cells because a cell specific promoter controls expression of the recombinase-LBD fusion protein. A strategy such as this has recently been used to induce the expression of diphtheria toxin in cultured cells (2).

Targeted expression of the diphtheria toxin gene (DT-A) to specific cells in transgenic mice results in death of those cells (4,36). Diphtheria toxin is an especially good choice for cell ablation studies because its mechanism of action is known (9), and the gene has been cloned, sequenced, and adapted for expression in mammalian cells. Naturally occurring diphtheria toxin is made by Corynebacterium diphtheriae as a secreted precursor polypeptide that is then enzymatically cleaved into two fragments, the A and B chains. The B chain binds to the surface of most eukaryotic cells and then delivers the A chain (DT-A) into the cytoplasm. Once inside the cell, DT-A inhibits protein synthesis. It is extremely toxic; a single molecule is sufficient to kill a cell (43). A DT gene, engineered for use in mammalian cells, DT-A, contains the coding sequence for the DT-A subunit, but not for the DT-B subunit (32). Cell-specific, cis-acting transcriptional regulatory elements drive expression of DT-A. The DT-A subunit is retained within the cytoplasm of the cell. In the absence of the B subunit, even DT-A released from dead cells is not able to enter other neighboring cells.

In the present invention, following androgen ablation therapy for the treatment of prostatic carcinoma, application of a regulated recombination system to target expression of DT-A to androgen independent cancer cells is employed as an effective way to prevent the development of recurrent tumors. In the normal prostate gland, the bcl-2 gene is expressed in androgen-independent basal cells (22,33). Bcl-2 expression is also associated with the appearance of androgen-independent prostatic tumors (10,28,33). Expression of the bcl-2 gene suppresses apoptosis in some cells, thereby contributing to the development, survival, and progression of hormone-refractory prostate cancer (37,38). It is not clear whether androgen-independent cells result from the selection of a subset of prostate cancer cells that were already hormone independent prior to androgen depletion, or whether gene expression changes in some cells as a consequence of hormone therapy. The promoters and other regulatory regions of the human bcl-2 gene have been cloned and are well-characterized (21). In the present invention, bcl-2 regulatory sequences are used to control expression of a recombinase-LBD fusion protein. The required genetic elements for this therapy are delivered to the diseased prostate of patients by means of newly developed viral vehicles.

The present invention also contemplates that the same strategy can be applied to the treatment of other cancers in which the bcl-2 gene is expressed, e.g., breast and colon cancer. Beyond that, by the judicious selection of the regulatory sequence controlling the expression of the recombinase-LBD fusion protein, this strategy can be adapted for the treatment of any cancer, for which the regulatory sequences of a molecular marker expressed in the cancer cell have been identified. Furthermore, this recombination system may be used for the targeted delivery of cytotoxins other than the A chain of diphtheria toxin (DT-A), e.g., ricin.

### SUMMARY OF THE INVENTION

The present invention is a method of treating prostate cancer in a mammal, comprising administering to said mammal a therapeutically effective amount of a regulated recombination system to target expression of DT-A to androgen-independent cancer cells.

Another aspect of the present invention is a method of treating cancer in a mammal, comprising administering to said mammal a therapeutically effective amount of a regulated recombination system to target expression of a toxin to cancer cells, wherein regulatory sequences of a molecular marker expressed in said cancer cells have been identified.

### BRIEF DESCRIPTION OF THE FIGURES

FIG. 1. The recombinase-LBD fusion protein is synthesized in cells but is not activated until the fusion protein translocates to the nucleus as a result of the ligand binding to the LBD. When the recombinase is active, site-specific recombination takes place and the target gene becomes functional. In this figure, the diphtheria toxin gene (DT-A) is the target gene.

FIG. 2. Regulated recombination system to target DT-A expression to bcl-2-expressing androgen independent cells. The FLP-ERt fusion protein is synthesized in bcl2-expressing cells but is not activated until administration of tamoxifen. When FLP is active, recombination occurs, rendering the DT-A gene functional.

FIG. 3. Induction of FLP recombinase mediated recombination results in DTA expression and cell death. Parental LNCAP cells (blue) and LNCaP cells stably transfected with the plasmid p22EDT1 (red) were cultured in the presence of 4-OHT of varying concentrations for 48 hours after which the trypan blue exclusion assay was used to assess cell viability. p22EDT1 contains an SV40 promoter driving an FLP-ERt gene sequence and a CMV promoter driving a DT-A sequence disrupted by an FRT-puro/pA-FRT cassette.

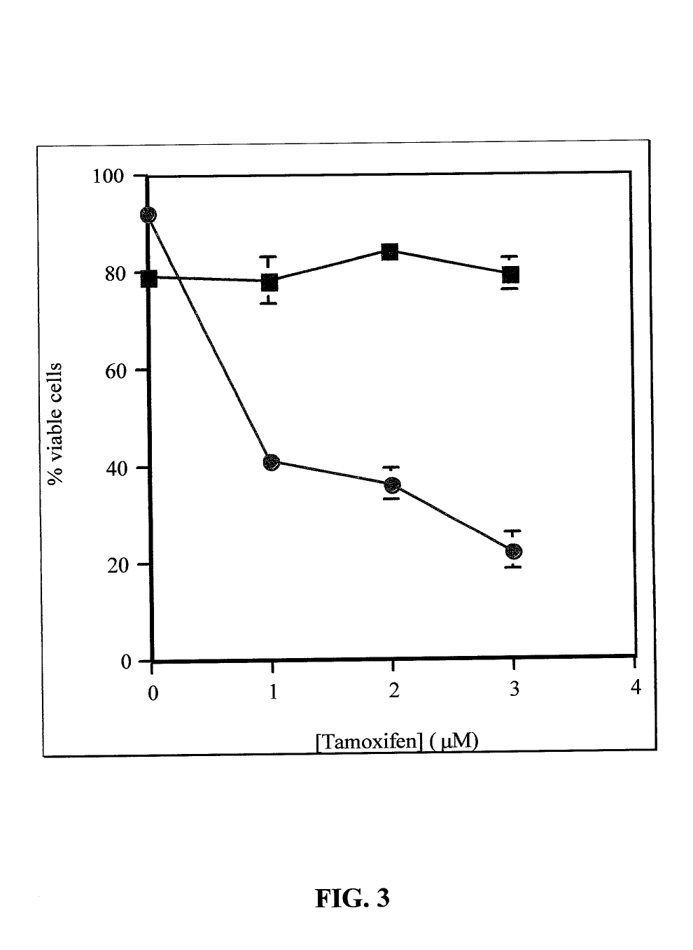

### DETAILED DESCRIPTION

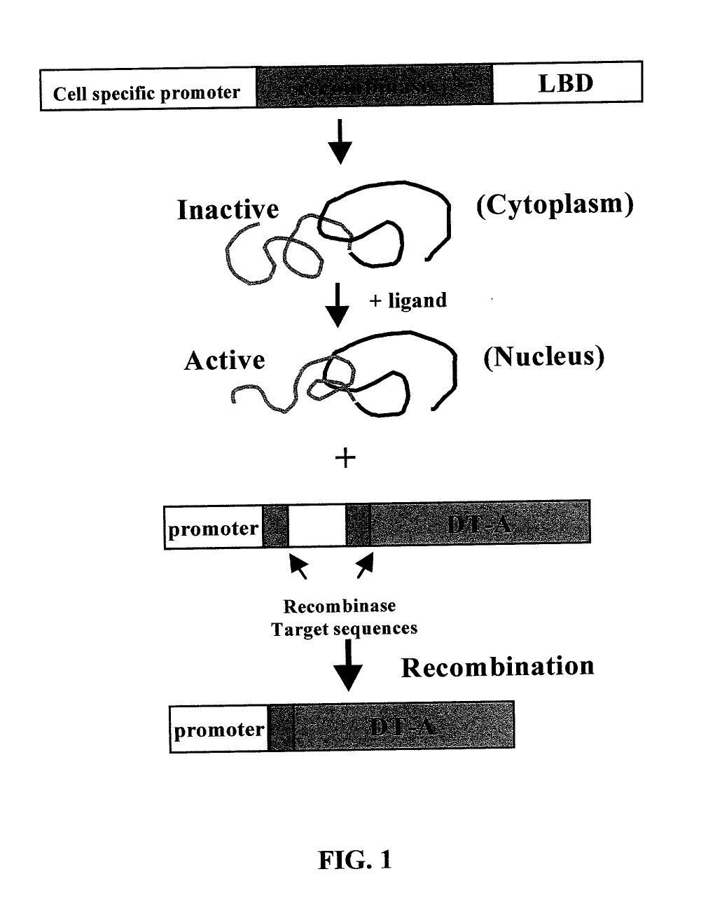

Targeted expression of diphtheria toxin A (DT-A) to androgen-independent prostatic cancer cells is an effective way to prevent the growth of recurrent androgen-independent tumors. In the present invention, an androgen independent human prostate cancer cell line, PC-3, is utilized. PC-3 cells were initiated from a grade IV prostatic adenocarcinoma. Using Western blot analysis, published reports that cells from this line express bcl-2 were confirmed (15,38). After introducing the genetic elements required of a regulated recombination system (see FIG. 1) into cultured PC-3 cells by adenoviral infection, the effects of exposure to ligand on viable cell number and cell growth were measured. To test the inducible, targeted expression of DT-A in bcl-2 expressing cells in vivo, adenoviruses containing the necessary elements for recombinase-mediated expression of DT-A in bcl-2-expressing cells are injected into PC-3-derived xenografts in nude mice. The effect of ligand administration on tumor size and cell growth is assessed. Furthermore, the same adenoviruses are injected directly into prostate tumors in castrated TRAMP (Transgenic Adenocarcinoma of Mouse Prostate) mice and the effect of ligand administration on tumor size and cell growth is determined.

Gene Constructs

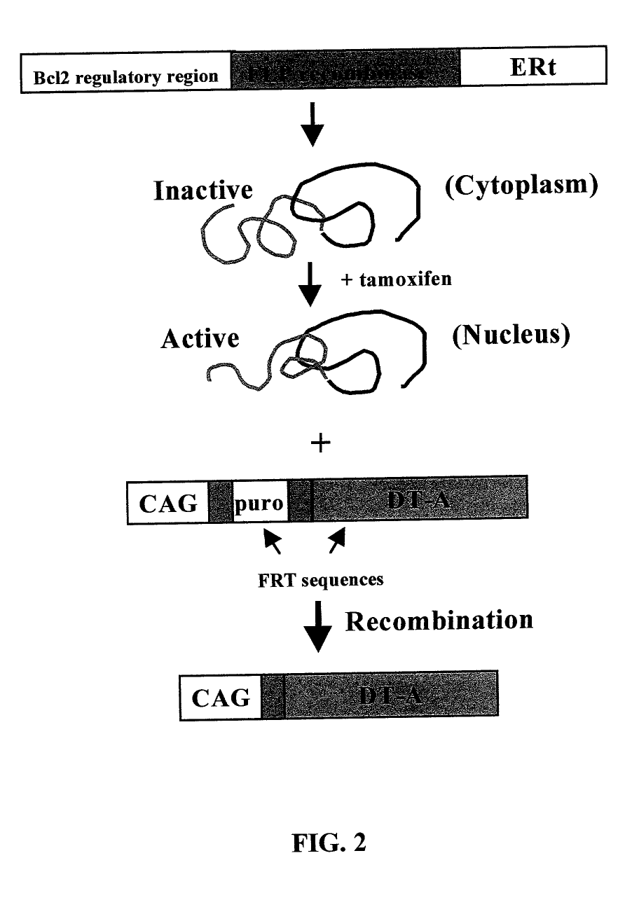

A gene encoding a fusion protein between FLP recombinase and a mutated ligand binding domain of the human estrogen receptor, ERt, is employed (FIG. 2). This fusion allows for the tamoxifen-dependent inducible activation of FLP. This fusion gene has been constructed using a plasmid, pCre-ERt, containing the modified estrogen receptor, obtained from Dr. Pierre Chambon (Pasteur Institute, Strasbourg, France). The fusion gene coding region is placed under the regulation of a 3.7 kb sequence upstream of the human bcl-2 gene shown to contain cis-acting elements required for the appropriate control of bcl-2 expression (21). Dr. John Reed (Burnham Institute, LaJolla, Calif.) supplied us with a plasmid, pTM449-2, containing this bcl-2 sequence.

The efficiency of FLP-mediated recombination is optimized by using a modified FLP sequence that has been shown to increase the frequency of recombination four-fold over that of the wild-type FLP (7). The plasmid, pOG-Flpe6, contains the modified FLP sequence (from Dr. A. Francis Stewart (EMBL, Heidelberg, Germany)).

A second construct, RSV/FRT₂puroDT-A, consists of the RSV promoter situated upstream of the DT-A coding sequence. The RSV promoter has been shown to be very active in human and dog prostate cells (1,29,30). Between the RSV promoter and the DT-A sequence is a puromycin coding sequence and a polyadenylation signal which blocks expression of the downstream DT-A (2). The puromycin gene is flanked by FLP recombination target (FRT) sites. Following binding of FLP to the FRTs, excisional recombination takes place resulting in positioning of the RSV promoter next to the DT-A sequence, thereby allowing for expression of DT-A (FIG. 2). Dr. A. Francis Stewart has supplied us with a plasmid, p22EDT1, containing the FRT-flanked puromycin and DT-A sequences (2).

A third construct, RSV/puro, is simply the RSV promoter driving the puromycin gene (not flanked by FRT sequences). This construct serves as a control in both in vitro and in vivo tests.

All gene sequences are housed in adenoviral expression vectors. This vector has been chosen over other vectors for the following reasons: 1. As compared to transfection with plasmids, adenoviruses infect most cells, delivering transgenes with high efficiency; 2. Adenoviruses infect both replicating and non-replicating cells, as compared to retroviruses which only infect replicating cells; 3. Recent studies by Lu and Steiner (29) identify important parameters in the use of adenoviruses for gene therapy in the prostate including the optimal route for viral delivery and the dissemination of the virus following delivery. 4. Adenoviral vectors have been used in other gene therapy strategies to deliver new genes to patients.

The AdMax™ System (Microbix, Toronto) is used for the efficient production of Ad5 viral vectors. Using this system, high efficiency site-specific recombination catalyzed by Cre recombinase results in rescue of the expression cassette into the left end of the El deleted Ad vector. The maximum size of the expression insert in these vectors is 7-8 kb (3). Therefore, given this constraint, the bcl-2/FLP-ERt (6.1 kb) and RSV/FRT₂puroDT-A (2.2 kb) sequences are housed in two different viruses. El-complementing 293 cells of low passage number are co-transfected with a genomic plasmid and a shuttle plasmid containing either bcl-2/FLP-ERt or RSV/FRT₂puroDT-A. Single viral clones are propagated and adenoviruses are purified from the culture medium of 293 cells showing a complete cytopathic effect. The viruses are concentrated twice by CsCl gradient ultracentrifugation and the viral titer determined by plaque assays in 293 cells (19). For in vitro tests, cells are co-infected with viruses carrying either the bcl-2/FLP-ERt or the RSV/FRT₂puroDT-A expression cassettes. For in vivo tests, viruses are injected directly into xenografts or into recurrent prostate tumors in TRAMP mice. Dr. Frank Graham has assured us that under the optimal virus-to-cell ratio (moi), close to 100% of cells will be infected with both viruses (personal communication).

In vitro Test of Gene Therapy System

PC-3 cells are co-infected with the bcl2/FLP-ERt and RSV/FRT₂puroDT-A containing adenoviruses. Cell death resulting from administration of the synthetic ligand 4-hydroxytamoxifen (4-OHT) is tested in cultured cells. Adenoviral infection is carried out by the addition of viral solutions of varying ratios of the two viruses and at varying degrees of multiplicity of infection to cell monolayers. After three hours of exposure to virus, the medium is changed and cells are incubated in fresh medium containing 4-OHT. Three concentrations of 4-OHT are tested: 1, 2, and 3 μM. After 48 hours incubation with 4-OHT, the number of viable cells are determined using the Trypan blue exclusion assay and the MTT assay (8). Since DT-A kills irreversibly by both apoptosis, as well as nonapoptotic pathways (39), the number of apoptotic cells is also determined. In addition, the growth potential of cells following treatment with tamoxifen is determined using a clonogenic assay (8).

In infected cells, the binding of 4-OHT to the FLP-ERt fusion protein will activate FLP. This in turn will result in recombinational excision of the FRT-puropA-FRT sequence, expression of the DT-A subunit, and cell death. Therefore, the number of viable and clonogenic cells in 4-OHT-treated cultures to be significantly reduced compared to the number of viable and clonogenic cells in non-treated cultures. Control infections are done with the RSV/FRT₂puroDT-A virus alone and with the RSV/puro virus. The viablity of cells infected only with the RSV/FRT₂puroDT-A construct serve as a control to measure the spontaneous excision of the puromycin cassette, an event which would result in expression of DT-A. The control in which cells are transfected with a RSV/puro construct serves to measure cell death resulting from 4-OHT toxicity.

In vivo Test of Gene Therapy System

Xenografts

To further test the efficacy of the regulated recombination system for targeting DT-A to bcl-2 expressing cells, xenografts are generated in male BALB/c nu/nu athymic mice by the subcutaneous injection of PC-3 cells (38,40,42). To generate tumors, 106 cells are mixed with Matrigel basement membrane matrix and injected subcutaneously into the flank of a mouse (38). When tumors reach a volume of about 50 mm³, a mixture of bcl2/FLP-ERt and RSV/FRT₂puroDT-A containing virus is injected directly into the tumor site. A 1:1 ratio of the two viruses is maintained, while the total number of plaque forming units varies (10⁹, 5×10⁹, and 10¹⁰). Twenty-four hours after injection of the virus, 4-OHT is administered to the host mice either by a single injection directly into the tumor (100 μl of a 3 μM solution in PBS) or by intraperitoneal injection on five consecutive days (1 mg in 100 μl sunflower seed oil). At one, two, and three weeks following the administration of 4-OHT, mice are euthanized, tumors are excised, and their volume and wet weight determined. Tumor volume is calculated by multiplying caliper measurements of length, width, and depth and then multiplying by 0.5236 (23). Tumor volume in the 4-OHT-treated test mice is significantly less than in control mice treated only with the vehicle. Following this procedure, the route of administration of 4-OHT, intratumoral or intraperitoneal, that is most effective at reducing tumor size is determined. Tumors are fixed and processed for histopathological examination. Other control groups in this study are xenografts infected only with the RSV/FRT₂puroDT-A virus and xenografts injected only with PBS. 4-OHT treatment of these mice has no effect on tumor size.

In vivo Test of Gene Therapy System

TRAMP Model

TRAMP mice contain the SV40 T antigen under the control of the prostate-specific probasin promoter (18,20). As a consequence of expression of this transgene, male TRAMP mice usually develop intraepithelial neoplasia (PIN) and/or well-differentiated prostate cancer by the time they are 10-12 weeks old. By 24-30 weeks of age, all TRAMP males develop prostate adenocarcinoma that metastasizes to distant sites, primarily lymph nodes and lungs. Following androgen ablation at 12 weeks of age, 70-80% of TRAMP males will develop androgen-independent disease (17), comparable to the emergence of hormone-refractory disease following androgen ablation therapy in prostate cancer patients. This animal model is used to test directly the specific killing of androgen-independent cells in recurrent tumors.

TRAMP mice, heterozygous for the PB-Tag transgene and maintained on a C57BL/6 background, are purchased from Jackson Laboratories. Female TRAMP mice are bred to non-transgenic male FVB mice to obtain transgenic (C57BL/6×FVB) F1 males. (TRAMP mice are more susceptible to developing prostate cancer when the transgene is on an FVB background). Transgenic mice are identified by PCR testing of genomic DNA from the tails of pups using primers as previously described (20). Twelve-week old TRAMP mice are castrated. Eight days later, when involution of the prostate tumor is well underway, a 1:1 mix of the bcl2/FLP-ERt and RSV/FRT₂puroDT-A viruses (total pfu=5×10⁹) is injected directly into the tumor. Lu et al. have previously shown that intratumoral injection seems to be the best route to treat local regional prostate cancer by viral-based gene therapy (29). To insure the accuracy of injections, mice are anesthesized and the prostate is imaged using an Acuson 128×P ultrasound unit having a 7 MHz linear probe. Similar to what is done with xenografts, 4-OHT is administered to the mice in either of two ways: 1. In one group, the vehicle for the viruses is phosphate buffered saline that contains 3 μM 4-OHT. Control injections do not contain 4-OHT. 2. In the other group, three days after injection of viruses into the prostate, mice receive the first of 5 consecutive day intraperitoneal injections of 4-OHT (1 μg/100 ml sunflower seed oil). Mice are euthanized and prostates are removed, weighed, and measured at 10 and 25 weeks after the viral injections. Tumors are also fixed and processed for histopathological examination. Similar to the controls for the xenograft experiments, control groups in this study are tumors infected only with the RSV/FRT₂puroDT-A virus and tumors injected only with PBS. To assess differences in tumor sizes among different experimental groups in both the xenograft and TRAMP tumor experiments, a twosided ANOVA is performed. A sample size of 4 mice for each treatment yields a power>95% and p=0.05.

### REFERENCES

1. Allay, J. A., Steiner, M. S., Zhang, Y., et al. 2000. Adenovirus p16 gene therapy for prostate cancer. World J. Urol. 18, 111-120.

2. Angrand, P. -O., Woodroofe, C. P., Buchholz, F., et al. 1998. Inducible expression based on regulated recombination: a single vector strategy for stable expression in cultured cells. Nucl. Acids Res. 26, 3263-3269.

3. Bett, A. J., Prevec, L. and Graham, F. L. 1993. Packaging capacity and stability of human adenovirus type 5 vectors. J. Virol. 67, 5911-5921.

4. Breitman, M. L., Clapoff, S., Rossant, J., et al. 1987. Genetic ablation: targeted expression of a toxin gene causes microphtalmia in transgenic mice. Science 238, 1563-1565.

5. Brocard, J., Feil, R., Chambon, P., et al. 1998. A chimeric Cre recombinase inducible by synthetic, but not by natural ligands of the glucocorticoid receptor. Nucleic Acids Res. 26, 4086-4090.

6. Brocard, J., Warot, X., Wendling, O., et al. 1997. Spatio-temporally controlled site-specific somatic mutagenesis in the mouse. Proc. Natl. Acad. Sci. USA 94, 14559-14563.

7. Buchholz, F., Angrand, P. -O. and Stewart, A. F. 1998. Improved properties of FLP recombinase evolved by cycling mutagenesis. Nature Biotech. 16, 657-662.

8. Carmichael, J., DeGraff, W. G., Gazdar, A. F., et al. 1987. Evaluation of a tetrazolium-based semiautomated calorimetric assay: assessment of chemosensitivity testing. Canc. Res. 47, 936-942.

9. Collier, R. J. 1975. Diphtheria toxin: mode of action and structure. Bacteriol. Rev. 39, 54-85.

10. Colombel, M., Symmans, F., Gil, S., et al. 1993. Detection of the apoptosis-suppressing oncoprotein bcl-2 in hormone-refractory human prostate cancers. Am. J. Pathol. 143, 390-400.

11. Culig, Z., Hobisch, A., Cronauer, M. V., et al. 1995. Activation of the androgen receptor by polypepetide growth factors and cellular regulators. World J. Urol. 13, 285-289.

12. Culig, Z., Hobisch, A., Cronauer, M. V., et al. 1994. Androgen receptor activation in prostatic tumor cell lines by insulin-like growth factor and epidermal growth factor. Cancer Res. 54, 5474-5478.

13. Culig, Z., Hobisch, A., Hittmair, A., et al. 1997. Androgen receptor gene mutations in prostate cancer. Implications for disease progression and therapy. Drugs Aging 10, 50-58.

14. Danielian, P. S., Muccino, D., Rowitch, D. H., et al. 1998. Modification of gene activity in mouse embryos in utero by a tamoxifen-inducible form of Cre recombinase. Curr. Biol. 8, 1323-1326.

15. Dorai, T., Perlman, H., Walsh, K., et al. 1999. A recombinant defective adenoviral agent expressing anti-bcl-2 ribozyme promotes apoptosis of bcl-2-expressing human prostate cancer cells. Int. J. Cancer 82, 846-852.

16. Furuya, Y., Krajewski, S., Epstein, J. I., et al. 1996. Expression of bcl-2 and the progression of human and rodent prostate cancers. Clin. Cancer Res. 2, 389-398.

17. Gingrich, J. R., Barrios, R. J., Kattan, M. W., et al. 1997. Androgen-independent prostate cancer progression in the TRAMP model. Canc. Res. 57, 4687-4691.

18. Gingrich, J. R., Barrios, R. J., Morton, R. A., et al. 1996. Metastatic cancer in a transgenic mouse. Cancer Res. 56, 4096-4102.

19. Graham, F. L. and Prevec, L. 1991. Manipulation of adenovirus vectors. Methods in molecular biology., Volume 7 (Clifton: Humana).

20. Greenberg, N. M., DeMayo, F., Finegold, M. J., et al. 1995. Prostate cancer in a transgenic mouse. Proc. Natl. Acad. Sci. U.S.A. 92, 3439-3443.

21. Harigai, M., Miyashita, T., Hanada, M., et al. 1996. A cis-acting element in the BCL-2 gene controls expression through translational mechanisms. Oncogene 12, 1369-1374.

22. Hockenbery, D. M., Zutter, M., Hickey, W., et al. 1991. BCL2 protein is topographically restricted in tissues characterized by apoptotic cell death. Proc. Natl. Acad. Sci. USA 88, 6961-6965.

23. Janek, P., Briand, P. and Hartman, N. R. 1975. The effect if estribe-progesterone treatment on cell proliferation kinetics of hormone-dependent GR mouse mammary tumors. Cancer Res. 35, 3698-3704.

24. Koivisto, P. A., Kononen, J., Palmberg, C., et al. 1997. Androgen receptor gene amplification: a possible molecular mechanism for androgen deprivation therapy failure in prostate cancer. Cancer Res. 57, 314-319.

25. Koivisto, P. A. and Rantala, I. 1999. Amplification of the androgen receptor gene is associated with P53 mutation in hormone-refractory recurrent prostate cancer. J. Pathol 187, 237-241.

26. Krajewska, M., Krajewski, S., Epstein, J. I., et al. 1996. Immunohistochemical analysis of bcl-2, bax, bcl-X and mcl-1 expression in prostate cancers. A,. J. Pathol. 148, 1567-1576.

27. Lidor, Y. J., Lee, W. E., Nilson, J. H., et al. 1997. In vitro expression of the diphtheria toxin A-chain under the control of human chorionic gonadotropin gene promoters as a means of directing toxicity to ovarian cancer cell lines. Am. J. Obstet. Gynecol. 177, 579-585.

28. Liu, A. Y., Corey, E., Bladou, F., et al. 1996. Prostatic cell lineage markers: emergence of BCL2⁺ cells of human prostate cancer xenograft LuCaP 23 following castration. Int. J. Cancer 65, 85-89.

29. Lu, Y., Carraher, J., Zhang, Y., et al. 1999. Delivery of adenoviral vectors to the prostate for gene therapy. Canc. Gene Ther. 6, 64-72.

30. Lu, Y. and Steiner, M. S. 2000. Transcriptionally regulated adenoviruses for prostate-specific gene therapy. World J. Urol. 18, 93-101.

31. Maxwell, I. H., Glode, L. M. and Maxwell, F. 1991. Expression of the diphtheria toxin A-chain coding sequence under the control of promoters and enhancers from immunoglobulin genes as a means of directing toxicity to B-lymphoid cells. Cancer Res. 51, 4299-4304.

32. Maxwell, I. H., Maxwell, F. and Globe, L. M. 1986. Regulated expression of a diphtheria toxin A-chain gene transfected into human cells: possible strategy for inducing cancer cell suicide. Cancer Res. 46, 4660-4664.

33. McDonnell, T. J., Troncoso, P., Brisbay, S. M., et al. 1992. Expression of the protooncogene bcl-2 in the prostate and its association with emergence of androgen-independent prostate cancer. Cancer Res. 52, 6940-6944.

34. Murayama, Y., Tadakuma, T., Kunitomi, M., et al. 1999. Cell-specific expression of the diphtheria toxin A-chain coding sequence under the control of the upstream region of the human alpha-fetoprotein gene. J. Surg. Oncol. 70, 145-149.

35. Nasu, Y., Timme, T. L., Yang, G., et al. 1998. Suppression of caveolin expression induces androgen sensitivity in metastatic androgen-insensitive mouse prostate cancer cells. Nature Med. 4, 1062-1064.

36. Palmiter, R. D., Behringer, R. R., Quaife, C. J., et al. 1987. Cell lineage ablation in transgenic mice by cell-specific expression of a toxin gene. Cell 50, 435-443.

37. Perlman, H., Zhang, X., Chen, M. -W., et al. 1999. An elevated bax/bcl-2 ratio corresponds with the onset of prostate epithelial cell apoptosis. Cell Death Differentiation 6, 48-54.

38. Raffo, A. J., Perlman, H., Chen, M. W., et al. 1995. Overexpression of bcl-2 protects prostate cancer cells from apoptosis in vitro and confers resistance to androgen depletion in vivo. Cancer Res. 55, 4438-4445.

39. Rodrigues, R., Lim, H. -Y., Bartkowski, L. M., et al. 1998. Identification of diphtheria toxin via screening as a potent cell cycle and p53-independent cytotoxin for human prostate cancer therapeutics. Prostate 34, 259-269.

40. Thalmann, G. N., Anezinis, P. E., Chang, S. M., et al. 1994. Androgen-independent cancer progression and bone metastasis in the LNCaP model of human prostate cancer. Cancer Res. 54, 2577-2581.

41. Vasioukhin, V., Degenstein, L., Wise, B., et al. 1999. The magical touch: genome targeting in epidermal stem cells induced by tamoxifen application to mouse skin. Proc. Natl. Acad. Sci. U.S.A. 96, 8551-8556.

42. Wu, T. T., Sikes, R. A., Cui, Q., et al. 1998. Establishing human prostate cancer cell xenografts in bone: induction of osteoblastic reaction by prostate-specific antigen-producing tumors in athymic and SCID/bg mice using LNCaP and lineagederived metastatic sublines. Int. J. Cancer 77, 887-894.

43. Yamaizumi, M., Mekada, E., Uchida, T., et al. 1978. One molecule of diphtheria toxin fragment A introduced into a cell can kill the cell. Cell 15, 245-250.

I claim:

1. A method of treating prostate cancer in a mammal, comprising administering to said mammal a therapeutically effective amount of a regulated recombination system to target expression of DT-A to androgen-independent cancer cells, wherein said recombination system comprises a fusion of a ligand binding domain (LBD) to a site-specific recombinase and Bcl-2 regulatory sequences control expression of a recombinase-LBD fusion protein.

2. A method of treating cancer in a mammal, comprising administering to said mammal a therapeutically effective amount of a regulated recombination system to target expression of a toxin to cancer cells, wherein said recombination system comprises a fusion of a ligand binding domain (LBD) to a site-specific recombinase and cancer-cell specific regulatory sequences control expression of a recombinase-LBD fusion protein.

3. The method of claim 2, wherein said regulatory sequences are Bcl-2 regulatory sequences,

4. The method of claim 2, wherein said toxin is ricin or DT-A.

5. The method of claim 2, wherein said cancer is one of the group of prostate cancer, breast cancer, and colon cancer.

6. The method of claim 2, wherein said cancer is at least one of the group of head and neck cancer, lung cancer, a mediastinal neoplasm, gastrointestinal tract cancer, genitourinary system cancer, testicular cancer, gynecologic cancer, leukemia, bone or soft tissue sarcoma, skin cancer, malignant melanoma, a central nervous system neoplasm, lymphoma, a paraneoplastic syndrome, endocrine cancer, benign or malignant mesothelioma, childhood cancer, metastatic cancer, an AIDSrelated malignancy, or cancer of an unknown primary site.
